SEQUENCE LISTING (1 sequence)

SEQ ID NO: 1 - DNA, 46 bp, Artificial
  Oligonucleotide
aattaatacgactcactatagggagattttttttttttttttttvn

(19) United States
(12) Patent Application Publication
(10) Pub. No.: US 2008/0161549 A1
(43) Pub. Date: Jul. 3, 2008

(54) METHOD OF PURIFYING NUCLEIC ACID MOLECULES USING PROTEINASE K

(75) Inventors: Mark D. Morris, Lynnwood, WA (US); Michael R. Meyer, Seattle, WA (US); Jon C. Marlowe, Bothell, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: ROSETTA INPHARMATICS LLC, Seattle, WA (US)

(21) Appl. No.: 11/940,934

(22) Filed: Nov. 15, 2007

Related U.S. Application Data
(63) Continuation of application No. 10/942,624, filed on Sep. 16, 2004, now abandoned.
(60) Provisional application No. 60/513,933, filed on Oct. 24, 2003.

(51) Int. Cl. C07H 21/00

(52) U.S. Cl. 536/25.42

(57) ABSTRACT

The present invention provides methods for purifying nucleic acid molecules, wherein each method includes the steps of: (a) synthesizing nucleic acid molecules in a reaction mixture; (b) contacting the nucleic acid molecules with a proteinase for a period of time sufficient to degrade protein in the reaction mixture; (c) applying the nucleic acid molecules treated in accordance with step (b) to a size-limiting filter so that at least some of the nucleic acid molecules are trapped on the filter; and (d) washing the filter with a phosphate buffer having a pH in the range of from about 5.7 to about 8.5.

### CROSS-REFERENCE TO RELATED APPLICATION

This application is a continuation of application Ser. No. 10/942,624, filed Sep. 16, 2004, which claims the benefit of Application No. 60/513,933, filed Oct. 24, 2003.

### FIELD OF THE INVENTION

The present invention relates to the purification of nucleic acid molecules.

### BACKGROUND OF THE INVENTION

Nucleic acid microarrays (hereinafter referred to as microarrays) are particularly useful in gene expression analysis at the level of transcription (see, e.g., Ramsay, Nature Biotechnol. 16: 40-44 (1998); Marshall and Hodgson, Nature Biotechnol. 16: 27-31 (1998); Lashkari et al., Proc. Natl. Acad. Sci. (USA) 94: 130-157 (1997); DeRisi et al., Science 278: 680-6 (1997)). In such analysis, the identity and abundance of a selected nucleic acid sequence in a sample is determined by measuring the level of hybridization of the nucleic acid sequence to probes on the microarray that comprise complementary sequences. The selected nucleic acid sequence in a sample can be an mRNA, or a nucleic acid molecule derived from an mRNA that has a nucleic acid sequence that is identical to, or complementary to, all, or a portion, of the mRNA. Using microarray expression assays, complex mixtures of labeled nucleic acids (e.g., mRNAs, or nucleic acid molecules derived from mRNAs) can be analyzed.

The analysis of gene expression using microarrays requires preparation of a sample that is hybridized to the microarray. The sample is typically a mixture of nucleic acid molecules that are identical to, or complementary to, mRNA molecules expressed in a particular type of cell. The nucleic acid molecules must be synthesized and labeled with a dye before being hybridized to a microarray. The synthesized nucleic acid molecules must be purified before they are hybridized to a microarray otherwise impurities (e.g., proteins and other organic molecules) interfere with the specific binding of the labeled nucleic acid molecules to the nucleic acid sequences attached to the microarray.

Many areas of biotechnology, such as drug discovery, increasingly rely upon the use of numerous microarrays to analyze gene expression in a large number of samples. For example, it may be necessary to screen thousands of candidate drugs for a desired biological activity by analyzing changes in gene expression caused by each of the drug candidates. Thus, each step of the process used to analyze gene expression should preferably be efficient, and inexpensive. Further, each step of the process used to analyze gene expression should preferably be amenable to automation so that many microarrays can be screened simultaneously or serially.

The present invention provides methods for purifying nucleic acid molecules that are relatively inexpensive to perform compared to the preferred purification methods currently used in the art. The methods of the present invention do not use noxious chemical reagents, and may be incorporated into a fully, or partially, automated system for analyzing gene expression.

### SUMMARY OF THE INVENTION

In accordance with the foregoing, the present invention provides methods for purifying nucleic acid molecules, wherein each method includes the steps of: (a) synthesizing nucleic acid molecules in a reaction mixture; (b) contacting the nucleic acid molecules with a proteinase for a period of time sufficient to degrade protein in the reaction mixture; (c) applying the nucleic acid molecules treated in accordance with step (b) to a size-limiting filter so that at least some of the nucleic acid molecules are trapped on the filter; and (d) washing the filter with a phosphate buffer having a pH in the range of from about pH 5.7 to about pH 8.5. The nucleic acid molecules may then be dried and resuspended in a buffer, or other solution. Proteinase K is an exemplary proteinase useful in the practice of the present invention.

In some embodiments of the method of claim 1, the nucleic acid molecules are cRNA, the proteinase consists essentially of proteinase K, and the phosphate buffer has a pH in the range of from about 5.7 to about 8.5.

The methods of the present invention are useful for purifying nucleic acid molecules for any purpose, such as the purification of cRNA or cDNA that is to be used to probe a microarray, or such as the purification of nucleic acid molecules that are to be attached to a substrate (e.g., silicon substrate) to form a microarray.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. Molecular Cloning: A Laboratory Manual, 2d ed., Cold Spring Harbor Press, Plainsview, N.Y. (1989) (hereinafter referred to as Sambrook et al.), and Ausubel et al., Current Protocols in Molecular Biology (Supplement 47), John Wiley & Sons, New York (1999), for definitions and terms of the art.

The present invention provides methods for purifying nucleic acid molecules, wherein each method includes the steps of: (a) synthesizing nucleic acid molecules in a reaction mixture; (b) contacting the nucleic acid molecules with a proteinase for a period of time sufficient to degrade protein in the reaction mixture; (c) applying the nucleic acid molecules treated in accordance with step (b) to a size-limiting filter so that at least some of the nucleic acid molecules are trapped on the filter; and (d) washing the filter with a phosphate buffer having a pH in the range of from about pH 5.7 to about pH 8.5. In some embodiments, the phosphate buffer has a pH in the range of from about 6.0 to about 8.0.

In the practice of the present invention, nucleic acid molecules are synthesized in a reaction mixture. Any type of nucleic acid molecule may be synthesized in the practice of the invention (e.g., complementary RNA (cRNA), complementary DNA (cDNA), and PNA, which is a DNA mimic having a peptide-like, inorganic backbone). As used herein, the term “nucleic acid molecule” encompasses modified nucleic acid molecules. Representative examples of modified nucleic acid molecules include nucleic acid molecules wherein one or more of the base moiety, sugar moiety and/or phosphate backbone is/are modified. For example, one or more nucleotides within a synthesized nucleic acid molecule may include one, or more, of the following modified base moieties: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, and 2,6-diaminopurine. Again by way of example, one or more nucleotides within a synthesized nucleic acid molecule may include one, or more, of the following modified sugar moieties: arabinose, 2-fluoroarabinose, xylulose, and hexose. Again by way of example, one or more nucleotides within a synthesized nucleic acid molecule may include one, or more, of the following modified phosphate backbones: a phosphorothioate, a phosphorodithioate, a phosphoramidothioate, a phosphoramidate, a phosphordiamidate, a methylphosphonate, an alkyl phosphotriester, and a formacetal. A synthesized nucleic acid molecule may also include an amino allyl derivatized uracil nucleotide. The amino allyl derivatized uracil nucleotide can be coupled, for example, via an aminoallyl linkage, to N-hydroxysuccinimide ester derivatives (NHS derivatives) of dyes (e.g., Cy-NHS, Cy3-NHS and/or Cy5-NHS).

Nucleic acid synthesis methods (e.g., in vitro transcription, and double strand cDNA synthesis) are well known in the art, and are described, for example, in the Sambrook et al. publication, and Ausubel et al. publication, supra.

After synthesis is complete, the synthesized nucleic acid molecules are contacted with at least one proteinase for a period of time sufficient to digest a portion, preferably most, more preferably all, of the protein present in the reaction mixture. Proteinases (sometimes also referred to as “proteases” in the art) are enzymes that digest proteins. The purpose of digestion with proteinase is to digest contaminating protein in the reaction mixture so that, when the reaction mixture is applied to a size-limiting filter, the digested protein fragments pass through the filter, while the synthesized nucleic acid molecules are retained on the filter.

Useful proteinases are preferably not contaminated with nucleases, or with nucleic acid molecules, and preferably have a molecular weight less than about 32,000 Daltons. Examples of proteinases useful in the practice of the invention include proteinase K (available, for example, from Sigma-Aldrich Corp., St. Louis, Mo., USA, and from Invitrogen, Carlsbad, Calif., USA), Subtilisin A (available, for example, from Novagen (EMD Biosciences, Inc., Novagen Brand, 441 Charmany Drive, Madison, Wis. 53719, USA), QIAGEN protease (available from Qiagen, 28159 Avenue Stanford, Valencia, Calif. 91355).

The time period during which the proteinase is incubated with the nucleic acid molecules depends, for example, on the number of units of proteinase added to the nucleic acid molecule reaction mixture, and the incubation temperature. Typically, however, sufficient proteinase is added to the reaction mixture so that an effective amount of protein degradation occurs within a period of time of between ten minutes and one hour.

The optimum incubation temperature depends on the proteinase. For example, proteinase K is most active at a temperature in the range of from 45° C. to 65° C. Vendors of particular proteinases provide information about the optimum temperature, and other reaction conditions that favor activity of the proteinase. The optimum reaction conditions for any proteinase can be readily determined by routine experimentation, if necessary.

Effective concentrations of proteinases useful in the practice of the present invention can readily be determined by one of ordinary skill in the art, and depend on such factors as the temperature and duration of incubation of the proteinase with the synthesized nucleic acid molecules. Thus, for example, one of ordinary skill in the art can test a range of proteinase concentrations in the practice of the present invention to determine which concentration range is most effective for each particular proteinase.

Examples of concentrations of proteinase K useful for digesting protein in the reaction mixture typically fall within the range of from 0.05 mg/mL to 1.0 mg/mL (e.g., from 0.125 mg/mL to 0.5 mg/mL).

By way of example, the nucleic acid molecule reaction mixture can be contacted with the proteinase by adding the reaction mixture to a dried aliquot of proteinase, and dissolving the proteinase in the reaction mixture. Again, by way of example, the reaction mixture can be contacted with the proteinase by adding an aliquot of a liquid proteinase solution to the reaction mixture.

After incubating the reaction mixture in the presence of a proteinase for a period of time effective to digest some, most, or all, of the protein present in the reaction mixture, the reaction mixture is applied to a size-limiting filter so that at least some, or all, of the nucleic acid molecules are trapped on the filter. As used herein, the phrase “trapped on the filter” means that molecules are trapped on the surface of the filter, or are trapped within the matrix of the filter. The size-limiting filter should be adapted to bind nucleic acid molecules having a chain length of at least 100 base pairs, but permit shorter nucleic acid molecules, or non-nucleic acid contaminants, such as protein fragments, to pass through the filter. Filters should preferably be free from nucleases. Examples of filters that are useful in the practice of the present invention include the following Millipore filters Millipore Montage PCR 96 Filter Plates (MANU 030), also known as the MultiScreen®-PCR Filter Plate (MANU 030); Millipore Montage PCR 384 Filter Plates (S384 PCR), and Millipore Montage PCR p-96 Filter Plates (LSKM PCR). The foregoing Millipore filters are available from Millipore, 290 Concord Rd., Billerica, Mass. 01821, USA. Again by way of example, Qiagen MinElute™96 UF PCR Purification Plates (available from Qiagen, 28159 Avenue Stanford, Valencia, Calif. 91355) are useful in the practice of the present invention. Also, for example, BD BioSciences Clontech Nucleofast® 96 PCR Plates (available from Clontech, 1020 East Meadow Circle, Palo Alto, Calif. 94303-4230) are useful in the practice of the present invention.

The nucleic acid molecules bound to the filter are washed at least once, preferably several times (e.g., between 2 and 10 times), with a phosphate buffer having a pH in the range of from about pH 5.7 to about pH 8.5. While not wishing to be bound by theory, the inventors currently believe that phosphate buffer in the foregoing pH range may interact with charged molecules (e.g., protein fragments) that are associated with the nucleic acid molecules, causing disassociation of the charged molecules therefrom. For example, potassium phosphate buffer, and sodium phosphate buffer, having a desired pH can be prepared as set forth in Appendix B.21 of Sambrook et al., supra.

After washing the filter with phosphate buffer, the filter may be dried, and the nucleic acid molecules resuspended in a buffer that is suitable for the next step in the process, such as labeling the nucleic acid molecules with a dye. Optionally, the dried filter may be washed with water to remove any residual phosphate ions that may interfere with a subsequent manipulation (e.g., labeling with dye) of the nucleic acid molecules.

The methods of the present invention may be performed manually (i.e., each step is performed by a human operator), or may be semi-automated (i.e., at least one step is performed by human operator, and the remaining step(s) is/are performed by a machine), or may be fully automated (i.e., all the steps are performed by at least one machine).

For example, the methods of the invention facilitate the use of the Fully Automated System (abbreviated as FAS system), for simultaneously synthesizing numerous nucleic acid probes, and using the probes to perform numerous hybridization reactions (e.g., screen numerous gene chips), which is disclosed in U.S. Provisional Patent Application Ser. No. 60/432,200, filed Dec. 10, 2002 (which application is incorporated herein by reference).

The following examples merely illustrate the best mode now contemplated for practicing the invention, but should not be construed to limit the invention.

### Example 1

This example describes a representative method of the invention for purifying nucleic acid molecules.

cRNA is produced in the following manner. The volume of a solution containing 5 μg of DNase-treated total RNA and 20 pmoles of primer T7T18VN (5′-AATTAATACGACTCACTATAGGGAGATTTTTTTTTTTTTTTTTTVN-3′, wherein N=any nucleotide, and V=G, A, or C) (SEQ ID NO:1) is increased to a volume of 10.5 μl with water. The RNA is denatured and the primer is hybridized to the mRNA by heating the sample to 65° C. for 10 minutes, followed by cooling to 4° C. for at least 5 minutes.

The following components are added to yield the concentrations listed in a final reaction mixture volume of 20 μl: 50 mM Tris pH 8.3, 75 mM KCl, 3 mM MgCl₂, 4 U/μl RNAGuard, 0.5 mM each dNTP (dATP, dCTP, dGTP, dTTP), 1.0 ng/μl random hexamers, 10 mM dithiothreitol, and 2.5 U/μl MMLV-RT. This reaction mixture is incubated for 2 hours at 40° C. to generate double stranded cDNA. The enzyme is then denatured by heating the reaction mixture at 65° C. for 15 minutes, followed by cooling to 4° C.

The total volume of the reaction mixture is increased to 80 μl with the following components at the concentrations listed: 40 mM Tris pH 7.5, 10.0 mM NaCl, 2.0 mM Spermidine, 14.25 mM MgCl₂, 200 U/mL RNAGuard, 2.5 mM each nucleotide triphosphate (ATP, CTP, GTP), 1.88 mM UTP, 0.6 mM amino-allyl UTP, 7.5 mM dithiothreitol, 25 kU/mL T7 RNA polymerase, and 15 U/mL inorganic pyrophosphatase. The in vitro transcription reaction is incubated for 16 hours at 40° C., followed by cooling to 4° C.

20 mL of distilled water is added to bring the reaction volume to 100 μL. Add the 100 μL of reaction mixture to lyophilized proteinase K (stored at −20° C.). The concentration of proteinase K is 0.25 mg/mL when dissolved in the reaction mixture. The lyophilized proteinase K is allowed to dissolve in the reaction mixture for 5 minutes at room temperature, and then the reaction mixture is robotically mixed by pipetting the mixture up and down several times.

The samples are briefly centrifuged, and the plates are sealed in thermal foil. The plates are then incubated for 30 minutes at 50° C. The plates are then placed into a Biomek® FX Robot for phosphate buffer multiscreen purification.

The samples are applied to a multiscreen filter (Millipore Multiscreen® PCR Filter Plate (MANU 030), available from Millipore, Billerica, Mass.), and subjected to a vacuum until dry. One hundred microliters of 0.1 M, pH 8.5, phosphate buffer is added to each well, and aspirated and dispensed ten times to redissolve the samples, and to wash the samples in the phosphate buffer. The samples are subjected to a vacuum until dry. 100 μL of a 0.1 M, Na₂HPO₄, pH 8.5, phosphate buffer is again added to each well, and the samples are resuspended therein. The samples are again subjected to a vacuum until dry.

100 μL of RNase-free water is added to each well, the sample is resuspended therein, and then subjected to a vacuum until dry. This process is repeated and then 100 μL of RNase-free water is added to each well, the samples are resuspended therein by aspiration and dispensing sixty times, and then transferred to a 96 well plate.

Thereafter, the samples may be coupled to a dye and used as probes to hybridize to a microarray.

### Example 2

This example shows the yield of cRNA obtained by using a purification method of the present invention compared to the yield of cRNA obtained by using an art-recognized, silica based, purification method.

cRNA was made from mRNA obtained from Jurkat cells, and purified using proteinase K and phosphate buffer as described in Example 1. Equal aliquots of a common pool of synthesized Jurkat cRNA was distributed into all the wells of two 96 well plates. The samples in one plate were purified in accordance with the present invention, while the samples in the other plate were purified using a standard silica binding purification method (RNeasy 96 kit, sold by Qiagen, 28159 Avenue Stanford, Valencia Calif. 91355). Vacuum settings for the filter purification were 18-22 inches Hg, and centrifugation for the silica binding method was carried out as per the manufacturer's recommendations. The results are shown in Table 1 below, wherein the abbreviation “StdDev” means standard deviation, the abbreviation “CV” means coefficient of variation, and N is the number of samples.

TABLE 1

| | Mean Yield | | | |
|---|---|---|---|---|
| cRNA Purification Method | (μg) | StdDev | CV | N |
| Purified by Silica Binding | 47.42 | 2.20 | 4.63 | 96 |
| Purified in accordance with present | 50.48 | 2.97 | 5.88 | 96 |
| invention | | | | |

As shown in Table 1, the yield obtained using a method of the present invention is slightly higher than the yield obtained using the prior art method (a p-value of 1.92×10⁻¹⁴ was obtained using a T-test, thus the two populations are statistically different at the 99% confidence level). The Coefficient of Variation (CV) shows the relative variation within each method (i.e., within each 96 well plate). As shown in Table 1, both methods have a desirably low CV across a full 96 well purification plate. Thus, there is little variation in the amount of cRNA obtained from each well in a plate. CV was calculated by dividing the standard deviation by the mean, and multiplying the quotient by one hundred. The product was rounded to the nearest hundredth.

### Example 3

This example shows that cRNA produced by a method of the present invention is a better probe for DNA microarrays than cRNA produced by an art-recognized silica binding method.

cRNA was produced from Jurkat cells, and from K562 cells, in 96 well plates. All of the individual Jurkat cRNA samples were pooled to form a Jurkat cRNA pool, and all of the individual K562 cRNA samples were pooled to form a K562 cRNA pool. The Jurkat cRNA pool was divided into 100 μl aliquots that were dispensed into replicate 96 well plates, and the individual samples were purified either in accordance with the present invention, or using an art-recognized, silica-based, purification method (RNeasy 96 kit, Qiagen, Valencia, Calif.). Similarly, the K562 cRNA pool was divided into 100 μl aliquots that were dispensed into replicate 96 well plates, and the individual samples were purified either in accordance with the present invention, or using an art-recognized, silica-based, purification method (RNeasy 96 kit, Qiagen, Valencia, Calif.).

The purified cRNA molecules were then coupled to cy3 or cy5 dye, and the samples were then filter purified to remove unincorporated dye. The dye-coupled samples were hybridized to Agilent flexjet 60mer oligonucleotide arrays containing approximately 25,000 different DNA sequences.

cRNA samples were purified each day, over a five-day period, so that 12 microarrays per purification method, per day, were hybridized (a total of 60 microarrays per method). One microarray outlier for the silica binding purification method was eliminated from the comparison due to an abnormally low gene signal to background ratio.

The results of the experiment are shown in Table 2, wherein the following abbreviations are used.

MdrG is the abbreviation for Maximum Dynamic Range Green, and is the ratio of the 90^{th} percentile green (cy3) intensity spots to either the 10^{th} percentile green intensity spots, or to the green background intensity (whichever is larger). MdrG provides a range of intensity that describes the sensitivity range of a method for the green (cy3) channel. MdrG is useful for analysis of both a same vs. same microarray (e.g., Jurkat vs. Jurkat), or a signature microarray (e.g., Jurkat vs. K562), because the intensity range of the green channel can be considered independently of fluor ratios. A method that produces a compressed Maximum Dynamic Range (Mdr) is less sensitive than a method that produces a broad Maximum Dynamic Range.

MdrR is the abbreviation for Maximum Dynamic Range Red, and is the same statistic as the MdrG except that MdrR applies to the red (cy5) channel.

GeneSbrG is the abbreviation for Gene Signal to Background Green and is an indicator of the signal to background ratio in the green (cy3) channel. GeneSbrG is a sensitive indicator of specific green channel intensity, and so is also an indicator of the purity of the cRNA (higher purity gives an improved signal to background ratio, and a higher GeneSbrG value), and the efficiency of cy dye coupling to the cRNA (higher efficiency of cy dye coupling gives an improved signal to background ratio, and a higher GeneSbrG value). GeneSbrG is the average green intensity per pixel of spot on the microarray (after background subtraction) divided by the standard deviation of the background for the green channel. GeneSbrG is useful for analysis of both a same vs. same microarray (e.g., Jurkat vs. Jurkat) or a signature microarray (e.g., Jurkat vs. K562) because the intensity range of the green channel can be considered independently of fluor ratios.

GeneSbrR is the abbreviation for Gene Signal to Background Red, and is the same statistic as the GeneSbrG except that GeneSbrR applies to the red (cy5) channel.

StdBkgG is the abbreviation for Standard Deviation of the Background Intensity in the Green (cy3) channel. StdBkgG is an indicator of the fluctuation in the background signal intensities. The background signals are the result of non-specific binding of material (e.g., labeled nucleic acid molecules, and contaminants such as proteins) on a microarray that causes fluorescent intensity. An increase in background fluctuation causes an increase in the value of StdBkG, and thus a decrease in the GeneSbrG which is calculated by dividing the average green intensity per pixel of spot on the microarray (after background subtraction) with the standard deviation of the background for the green channel. StdBkgG is useful for analysis of both a same vs. same microarray (e.g., Jurkat vs. Jurkat), or a signature microarray (e.g., Jurkat vs. K562), because the intensity range of the green channel can be considered independently of fluor ratios.

StdBkgR is the abbreviation for Standard Deviation of the Background Intensity in the Red (cy5) channel, and is the same statistic as the StdBkgG except that StdBkgR applies to the red (cy5) channel.

CV is the abbreviation for coefficient of variation. N is the number of samples.

TABLE 2

| | Silica Binding | | Purification in Accordance with | | |
|---|---|---|---|---|---|
| | Purification | | the Present Invention (N = 60) | | |
| | (N = 59) | | | | T-Test |
| | Mean | CV | Mean | CV | Pvalue |
| Gene_SbrG | 21.26 | 19.47 | 22.13 | 19.41 | 0.1744 |
| Gene_SbrR | 18.74 | 29.13 | 19.37 | 22.22 | 0.3173 |
| MdrG | 53.90 | 19.12 | 59.27 | 20.87 | 0.0154 |
| MdrR | 203.49 | 22.70 | 207.53 | 20.19 | 0.7615 |
| StdbkgG | 1.2 × 10⁻⁴ | 22.98 | 1.13 × 10⁻⁴ | 17.66 | 0.1392 |
| StdbkgR | 1.19 × 10⁻⁴ | 26.17 | 1.01 × 10⁻⁴ | 10.41 | 0.0002 |

The data set forth in Table 2 show that, over a significantly large set of microarrays, the hybridization performance of cRNA samples prepared using a purification method of the present invention is comparable, or better, than the hybridization performance of cRNA samples prepared using the industry standard, silica-binding, purification method.

For example, the values for StdbkgR for the cRNA samples purified using a method of the present invention are significantly lower than the corresponding values for the cRNA samples purified using an art-recognized, silica-based, purification method (i.e., there is less background variation when screening a DNA microarray using cRNA samples purified using a method of the present invention than when using cRNA samples purified using an art-recognized, silica-based, purification method).

Also, for example, the values for MdrG for the cRNA samples purified using a method of the present invention are significantly higher than the corresponding values for the cRNA samples purified using an art-recognized, silica-based, purification method (i.e., the sensitivity range obtained using cRNA samples purified using a method of the present invention is greater than the sensitivity range obtained using cRNA samples purified using an art-recognized, silica-based, purification method).

### Example 4

This example shows that the combination of proteinase K treatment and washing with phosphate buffer yields a higher quality cRNA sample, as assessed by hybridization to a DNA microarray, than treatment with proteinase K without subsequent washing using phosphate buffer. This example also shows that purification of cRNA using a method of the present invention yields a purified cRNA sample that is at least as good as a cRNA sample that was purified by an art-recognized silica-binding method, as assessed by the quality of the signal obtained when the purified cRNA samples are hybridized to a DNA microarray.

cRNA was synthesized from Jurkat cell mRNA, and from K562 cell mRNA as described in Example 1. The cRNA samples were treated with two different concentrations of proteinase K (0.05 mg/mL, and 0.5 mg/mL) for 10 minutes at 50° C. During filter purification (using a Millipore Multiscreen® PCR Filter Plate (MANU 030)), in accordance with the present invention, the cRNA was either washed with water only, or with two washes of 0.1 M sodium phosphate buffer (pH 8.5), followed by another two washes with water. The silica-binding purification method (using an RNeasy 96 kit, in accordance with the manufacturer's instructions) was used as the control, and as a comparison measure for the effectiveness of the combination of the proteinase K and phosphate buffer wash.

The purified cRNA was coupled to cy dye, filter purified using a Millipore Multiscreen® PCR Filter Plate (MANU 030), (available from Millipore, Billerica, Mass.), and hybridized to Agilent flexjet oligonucleotide arrays containing approximately 25,000 different DNA sequences. The results are shown in Table 3. The abbreviations GeneSbrR and GeneSbrG are described in Example 2. The GeneSbrR and GeneSbrG values were calculated from data obtained from eight single microarrays per purification method.

The abbreviation “PK” means proteinase K.

“Total Regulations” refers to the total number of genes that show statistically significant differential regulations (p-value <0.01) in a comparison of a Jurkat vs. K562 Fluor Reversed Pair (FRP), with N=4 FRPs for each purification method.

The Total Regulations statistic is a measure of the sensitivity of a hybridization method, by assessing the ability of a labeled nucleic acid sample to detect significant differential gene expression ratios. For example, when comparing labeled cRNA samples that are identical, except that one sample was purified using a proteinase and a phosphate buffer, whereas the other sample was purified using an art-recognized silica-based method, the Total Regulations value for both cRNA samples should, in principle, be the same (or any difference should not be statistically significant), since the hybridization pattern on the DNA microarray should be identical for each sample. In this “same vs. same” type of comparison, the Total Regulations statistic is a measure of the number of false positive hybridization signals, and so, when comparing two sample purification methods applied to identical nucleic acid samples, the method that gives the lower Total Regulations value is preferred. Conversely, a large Total Regulations value is desirable for a comparison between the hybridization patterns obtained using labeled cRNA samples from two different sources (e.g., Jurkat cells and K562 cells), since the higher Total Regulations value shows that the hybridization experiment is detecting more gene expression differences.

TABLE 3

| | Total | | | | | | | | |
|---|---|---|---|---|---|---|---|---|---|
| | Regulations | | T-test | GeneSbrR | | T-test | GeneSbrG | | T-test |
| Purification Process | Mean | StdDev | Pvalue | Mean | StdDev | Pvalue | Mean | StdDev | Pvalue |
| Silica Binding | 8287 | 69 | | 19.0 | 4.7 | | 16.1 | 1.8 | |
| 0.05 mg/mL PK | 7647 | 267 | 0.128 | 17.6 | 1.6 | 0.114 | 16.5 | 1.9 | 0.024 |
| 0.05 mg/mL PK and | 7907 | 125 | | 19.9 | 3.5 | | 19.7 | 3.1 | |
| Phosphate Wash | | | | | | | | | |
| 0.5 mg/mL PK | 7637 | 442 | 0.022 | 15.3 | 1.7 | 0.003 | 14.5 | 2.4 | 0.001 |
| 0.5 mg/mL PK and | 8321 | 66 | | 20.6 | 4.4 | | 21.7 | 3.9 | |
| Phosphate Wash | | | | | | | | | |

For each proteinase concentration, the Student t-test was used to compare the results obtained from samples purified using the combination of proteinase K and phosphate buffer with the results obtained from samples purified using proteinase K only results. At the 95% confidence interval, there was a significant difference in both the GeneSbrR and GeneSbrG channels for the 0.5 mg/mL proteinase K concentration versus the combination of 0.5 mg/mL proteinase K and phosphate buffer wash, thereby demonstrating that the combination of proteinase K treatment and phosphate buffer washing yielded greater hybridization sensitivity than the proteinase K treatment alone.

### Example 5

This example describes the determination of a useful concentration range of proteinase K to include in an embodiment of the methods of the present invention.

cRNA was made from total RNA from Jurkat and K562 cells in accordance with the present invention, and treated using the amounts of proteinase K shown in Table 4. Up to 56.2 μg Jurkat cRNA was treated with proteinase K, and up to 62.8 μg K562 cRNA was treated with proteinase K. The Jurkat cRNA and the K562 cRNA were each separately labeled with cy3 and cy5, to create Fluor Reversed Pairs for each cRNA sample. Then the labeled samples were filter purified to remove the unincorporated dye, and then hybridized onto Agilent flexjet oligonucleotide microarrays containing approximately 20,000 different DNA sequences.

The “Total Regulations” statistic is described in Example 4 and is a measure of the total number of genes that show statistically significant differential regulations (p-value <0.01) in a Jurkat vs. K562 Fluor Reversed Pair (FRP) comparison. For each concentration of proteinase K, there were two FRPs (4 microarrays total).

To calculate the Standard Deviation of the Background Red and Green (StdBKgR and StdBKgG) 8 microarrays for each concentration of proteinase K were compared.

As shown in Table 4, at a concentration between 0.125 mg/mL through 0.75 mg/mL, proteinase K treatment produced low noise (low StdBkg value) on the microarrays. There was a 2-3-fold increase in the background noise when proteinase K was not used. This increase in background results in decreased sensitivity, as shown by the lower value for the Total Regulations for the 0 mg/mL proteinase K treatment. A concentration of proteinase K greater than 0.75 mg/mL increased the filtration time, perhaps due to clogging of the filter (data not shown). Based on the results of this experiment, a proteinase concentration between 0.125 mg/mL and 0.5 mg/mL is regarded as an effective concentration for purifying up to 60 μg proteinase K in this embodiment of the present invention.

TABLE 4

| | | Total Regulations | | StdBKgR | | StdBKgG | |
|---|---|---|---|---|---|---|---|
| | | (N = 2 FRPs) | | (N = 8) | | (N = 8) | |
| Proteinase K | | Mean | StdDev | Mean | StdDev | Mean | StdDev |
| 0 | mg/mL | 5294 | 363 | 3.57 × 10⁻⁴ | 9.07 × 10⁻⁵ | 2.68 × 10⁻⁴ | 7.81 × 10⁻⁵ |
| 0.125 | mg/mL | 8417 | 69 | 9.00 × 10⁻⁵ | 3.93 × 10⁻⁶ | 7.50 × 10⁻⁵ | 4.75 × 10⁻⁶ |
| 0.25 | mg/mL | 8838 | 27 | 1.18 × 10⁻⁴ | 1.03 × 10⁻⁵ | 9.64 × 10⁻⁵ | 1.13 × 10⁻⁵ |
| 0.5 | mg/mL | 7776 | 191 | 1.10 × 10⁻⁴ | 1.43 × 10⁻⁵ | 9.33 × 10⁻⁵ | 1.41 × 10⁻⁵ |
| 0.75 | mg/mL | 8238 | 180 | 9.71 × 10⁻⁵ | 6.20 × 10⁻⁶ | 9.30 × 10⁻⁵ | 6.89 × 10⁻⁶ |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of purifying nucleic acid molecules, said method comprising the steps of:
(a) synthesizing nucleic acid molecules in a reaction mixture;
(b) contacting the nucleic acid molecules with a proteinase for a period of time sufficient to degrade protein in the reaction mixture;
(c) applying the nucleic acid molecules treated in accordance with step (b) to a size-limiting filter so that at least some of the nucleic acid molecules are trapped on the filter; and
(d) washing the filter with a phosphate buffer having a pH in the range of from about 5.7 to about 8.5.

2. The method of claim 1 further comprising the step of drying the nucleic acid molecules treated in accordance with step (d) on the filter and resuspending the dried nucleic acid molecules in an aqueous solution.

3. The method of claim 1 wherein the nucleic acid molecules are cRNA.

4. The method claim 1 wherein the nucleic acid molecules are cDNA.

5. The method of claim 1 wherein the nucleic acid molecules are contacted with the proteinase by dissolving a dried proteinase in the reaction mixture.

6. The method of claim 1 wherein the nucleic acid molecules are contacted with the proteinase by adding an aliquot of proteinase solution to the reaction mixture.

7. The method of claim 1 wherein the phosphate buffer has a pH in the range of from about 6.0 to about 8.0.

8. The method of claim 1 wherein the proteinase is selected from the group consisting of proteinase K and Subtilisin A.

9. The method of claim 8 wherein the proteinase consists essentially of proteinase K.

10. The method of claim 1 wherein the nucleic acid molecules are contacted with the proteinase for a period of time of between ten minutes and one hour.

11. The method of claim 1 wherein the nucleic acid molecules are cRNA, the proteinase consists essentially of proteinase K, and the phosphate buffer has a pH in the range of from about 5.7 to about 8.5.

12. The method of claim 1 wherein the method is automated.
